(19) United States
(12) Patent Application Publication
(10) Pub. No.: US 2022/0176113 A1
(43) Pub. Date: Jun. 9, 2022

(54) COSMIC RESONATOR

(71) Applicants: Ali Akbar MOHADDES, Tehran (IR); Marzieh AFSHARI CHAMANABADI, Blvd - Mashhad (IR); Saeid SAATCHI, Hopkins, MN (US); Mohammadali ASSTCHI, Mashhad (IR)

(72) Inventors: Ali Akbar MOHADDES, Tehran (IR); Marzieh AFSHARI CHAMANABADI, Blvd - Mashhad (IR); Saeid SAATCHI, Hopkins, MN (US); Mohammadali ASSTCHI, Mashhad (IR)

(21) Appl. No.: 17/601,138

(22) PCT Filed: Apr. 20, 2019
(86) PCT No.: PCT/IB2019/053287

§ 371 Date: Oct. 4, 2021

(51) Int. Cl. A61N 1/16; A61G 10/00

(52) U.S. Cl. CPC A61N 1/16; A61G 10/00

(57) ABSTRACT

A cosmic resonator is designed and constructed to trap the cosmic waves acting as a quantum trap the upper half of which is to absorb and the lower half is increase resonance and to increase the durability of input cosmic waves. The enhanced cosmic waves within the pyramid are used to load vitality force information on water or any water-based creature or matter with direct human exposure to cosmic waves and indirect gain with consumption of cosmic energetic products that are exposed to the waves to improve physical and mental health of humans.

As the cosmic wavelength is higher than gamma waves, trapping it in more than one second time requires a very detailed, technical and optical design. In this invention, a pyramid resonator is designed and constructed to trap the cosmic waves acting as a quantum trap the upper half of which is to absorb and the lower half is increase resonance and to increase the durability of input cosmic waves. The result of using this pyramid quantum super cavity is the use of this energy to bring vitality force data onto water or any living creature or substance formed of water by two methods of direct human exposure to waves and indirect gain of cosmic waves consuming cosmic energetic products for the physical and mental health.

### TECHNICAL FIELD

It is generally related to the cosmic waves, pyramid, pyramid therapy and physical and mental health.

### BACKGROUND ART

Description or the related art including information disclosed. examples of such assemblies are disclosed in the following U.S. Pat. Nos.:

1—PCT application No: WO2015022697A1 discloses the house has a tool for optimal placement based on a topic in a health area. The inventor by creating a space that combines a rectangular cube on the bottom and a copper pyramid roof enables the individual to enjoy meditation and cosmic relaxation by combining several subjects.

By combining alternative therapies (complementary medicine) in an “enclosed home”, the authors claim the following issues in health area:

The use of aromatic compounds for aromatherapy

Gem therapy and use of quartz crystals acting as a kind of cosmic wave storage

Since each chakra has certain color and halo, 7 colors of the rainbow are used to set the chakras.

Enjoying electromagnetic radiation

Using pure oxygen

Taking advantage of the GY energy reflexes or the CHI Prana inside the pyramid

Similarities and differences with the proposed invention:

In the proposed “cosmic resonator pyramid” invention the design principles are based on absorption, reflection, increased vibration and maximum, preservation and survival of cosmic waves as a pyramid optical cavity according to the maps. In its structure, the use of any irrelevant material in the form of multiple demands collapses the waveguide base and reduces the efficiency of the device.

The “Etheric Cosmic Resonator” is distinct from the above-mentioned invention in terms of design, structure, material, angles, and essentially the geometric shape.

The cosmic resonator does not utilize gem therapy mechanism, pure oxygen, light therapy and aromatherapy.

2—Hahn, Matthias, DE application No: DE102007010424 discloses the inventor has used Quartz and crystalline rocks to load cosmic information on water and juice.

In fact, these rocks act like a cosmic wave protector; therefore, the granites are used in temples and Khufu's temple.

If one cubic centimeter of granite stones is pressed, they present electric output.

However, energizing water and juice is one of products and outputs of this invention. Here loading information and energizing is not done by stone and it is performed based on exposing water or thing that is formed of water to the cosmic waves.

3—Geissmann, Robert, EP Application No: EP19800400320 discloses the invention is merely a pyramid structure with observation of the maps, which is used in part of pyramid sections and is a fireplace.

4—Li Yuanliang, CN patent No: CN1617188 discloses the Pyramid energy focusing model and its making method.

In the model, the energy concentration pyramid is made of plastics, plywood, fiberboards, granites and other insulating materials in the form of a rectangular or triangular pyramid with of each side as a smooth triangle. However, none of these materials is used in our invention.

Part of the designed pyramid is taken from Egyptian Pyramids with materials such as plastic, plywood, ceramic, wood or insulating materials such as granite and limestone and it is made of four-sided square or a cone-shaped pyramid. None of these materials is used in our invention and there is a similarly in terms of 52° angle.

### SUMMARY OF INVENTION

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes cosmic resonator. It is in the technical field of cosmic waves, pyramid, pyramid therapy and mental and physical health that pyramid resonator is built with the ability to trap cosmic waves as a quantum trap. They have the absorption feature, increases resonance and durability of input cosmic waves with the aim of converting energy and dark matter, benefiting from frequency wavelengths and quantum space information to expand the boundaries of medical, engineering and basic sciences.

One of its outputs in the health sector is the loading of vital information on the subatomic structure of materials including water and anything formed of water for human health in the form of homeostasis energy balance. Connecting the etheric body to the cosmic intelligence network, which results in treating mental and neuronal intelligence illnesses and all illnesses related to cellular intelligence and reducing the length of treatment after a variety of transplantational surgeries in humans.

The previous, traditional and non-scientific pyramid treatment methods were based on using various types of rock materials, in particular granite, wood, glass, and other non-related basic metals that caused the deviation of cosmic waves rather than conducting and concentrating these waves.

At small scale, engineering practices are made to convert ground energy into electrical energy or to use the pyramid's reflective environments through sources of Tesla coil artificial generators for generating scalar waves or at very low scales such as radio waves. No artificial sources have been used in the present invention.

As the cosmic wavelength is higher than gamma waves, trapping it in more than one second time requires a very detailed, technical and optical design. In this invention, after simulating and examining a large amount of materials and different angles to trap these waves for more than an hour and maximizing cosmic wave in the cavity up to 21 times id due to the use of this energy along the loading vitality force and etheric body in this pyramid quantum cavity.

These waves are used to load vitality force information on water and anything made of water such as fruits, vegetables, livestock and fish and energetic cosmetic products indirectly and directly by exposing the patient in the energy field. The presentation method is as energy products, and the production and sale of pyramid at specified scales and the provision of services as a project in the form of the implementation of a set of pyramid networks as Cosmic Health Land.

### Technical Problem

Since pyramid therapy has 5000-year background, the humankind uses the cosmic waves inside the pyramid for physical and mental health. Cosmic rays are mysterious radiations conveyed through the space constantly. They enter the atmosphere by the electrically charged subatomic particles. Since cosmic rays are electrically charged, they are diverted by various magnetic fields across the galaxy. Because of this deviation, cosmic rays do not return to their source directly and they have not been identified yet. What is important here is not the origin of cosmic rays, but the natural effects of them on subatomic layers. These rays have a much higher energy and frequency than gamma rays.

The most powerful energy and frequency detected in the universe are cosmic waves whose energies are in the range of {10⁸ ev to 10²⁰ ev} their frequency range is {10²¹ HZ to 10ⁿ HZ} and their wavelength in landa (m) is {10-12 [symbol] m to 10-22 [symbol] m}. It also has the ability to pass through any material in the finest atomic layers. Therefore, trapping of cosmic waves is a very scientific and complex subject, which causes the pyramid structures to be neglected in optical and reflection coefficients studies.

Consequently, despite the amazing therapeutic effects, pyramid structures have not been capable of keeping cosmic waves within more than one second. (Components at their most basic level are not chemical reactions, but an energetic load). The humankind and living organisms are a combination of energy in the energy field that everything in the world is connected to another one.

In the last 50 years, rehabilitation of patients with CP and MS, various types of diseases like cancers, Parkinson's, diabetes and AIDS and various types of infertility has been developed. One of the most important problems is that the artificial human waveforms called technology carry destructive information that lead cellular and neurological insanity that the body's defense system is virtually unavailable and unable to deal with them. At low doses, the body cells can restore many of the injuries inflicted by the body by radiation. At doses greater than 100 mSv/h the protective mechanisms of cells do not have the chance to repair all the injuries. In this case, the severity of the effects increases nonlinearly and causes the patient to develop mental and physical illnesses.

The previous, traditional and non-scientific pyramid treatment methods were based on using various types of rock materials, in particular granite, wood, glass, and other non-related basic metals that caused the deviation of cosmic waves rather than conducting and concentrating these waves.

At small scale, engineering practices are made to convert ground energy into electrical energy or to use the pyramid's reflective environments through sources of Tesla coil artificial generators for generating scalar waves or at very low scales such as radio waves.

In recent years, various studies have been done on gamma rays and their effects on the biological structure of the human body.

Gamma ray when it passes through the material collides with its atoms and sometimes because of these collisions, electrons are separated from the atoms of these materials and ions are produced. Due to the collision of gamma ray with humans, it passes through the cellular structure and destroys deoxyribonucleic acid or DNA. Cosmic rays have higher energy of gamma rays and affect the subatomic structure, but the source of these rays is still unknown. Kudela (2009) estimated the energy range of these rays as 10⁹ ev to 10²⁰ ev. The energy and frequency spectrum of these rays is in phase with the biological structures of the organisms and does not have the impacts of other frequencies.

One of the most powerful and effective solutions to this problem is “Frequency Feng Shui with Cosmic Waves” which carries basic information called the Vitality force, provided that these waves are trapped and exploited for the health of the human being.

### Invention Objectives

Energizing the material based on the absorption of cosmic waves at three maxima inside the pyramid with the mentioned materials including water or anything that is made up of water, construction of Cosmic Health Land and establishment of the pyramid at 1, 10 and 100 scale in parallel in order to achieve the following goals:

Energizing the livestock products by cosmic waves by deploying them at the center of the pyramid to expose materials to cosmic waves in the pyramid for about 40 days

Energizing fish and vegetables by the cosmic waves in the water of the pyramid for 21 days in the form of fish culture in energetic water

Production of vegetables in the water of the pyramid and the human use of these energetic and cosmetic products for physical and mental health

The production of life elixir and structural H3O2 at the pyramid vertex within the spherical orb for 7 days for cellular rejuvenation

Producing energetic cosmetic products by exposing these products to cosmic waves for 7 days at the pyramid vertex inside the spherical orb and benefiting from it to eliminate skin damages

Breeding livestock in the pyramid and energizing animal products with cosmic waves by exposing at the center of the pyramid for 40 days and benefiting from the meat and dairy in order to achieve physical and mental health.

Producing energetic water to maintain human health

The presence of humans in the maximum of one-third of the pyramid is due to physical and psychological health (and frequency feng shui due to cosmic waves.

Human bath in the energetic water at the bottom of pyramid with 1 or 10 or 100 scale and connecting the body to vitality force and treatment of illnesses related to cellular and neuronal intelligence as well as loading vital information on the subatomic structure of materials including water and anything formed of water for human health in the form of homeostasis energy balance. Connecting the etheric body to the cosmic intelligence network, which results in treating mental and neuronal intelligence illnesses and all illnesses related to cellular intelligence and reducing the length of treatment after a variety of transplantational surgeries in humans.

### Solution to Problem

Since wave transmits information, cosmic waves carry vital force information and on the other hand, the advent of technology and the use of wavelengths with radiant doses above 100 mSv cause free radicals in the cell that lead to the emergence of mental illness, neuronal damage and illnesses associated with cellular intelligence, the use of Vital Force for the health of humans, water and any water-based organism is a decisive and practical solution for the treatment of diseases associated with cellular intelligence and neuronal intelligence. Accordingly the mission of the invention is to create a pyramid resonator with the ability to trap cosmic waves, which acts as a quantum trap that balances the body homeostasis in the body's etheric layer.

The process of producing energy products by the materials, including water or anything that is made of water, and human exposure to cosmic waves simultaneously by simultaneous and parallel deployment of pyramids with 1 or 10 or 100 scale as a place called cosmic health results in using the energetic products and human exposure to cosmic waves;

Rehabilitation of all types of CP, MS and Parkinson's diseases

Treating various types of cancer and illnesses associated with cellular intelligence, including AIDS

Treating various neuronal neurological diseases

Reducing the duration of treatment after all types of surgeries and transplantation

Trapping cosmic waves for more than an hour in a quantum pyramid cavity and benefiting from it for the treatment of physical and mental illnesses

Maximizing cosmic wave in the cavity up to 21 times without the presence of the patient

Maximizing cosmic wave in the cavity up to 50 times with the presence of the patient

Since these life based waves are the energy of all creatures, exposure to these waves has no harmful physiological and psychological effects

Molecular rehabilitation and youth renewal known as elixir of life

Modifying genetic codes in DNA

Resolving infertility crisis

Definitive treatment of diabetes

Production of energy products, including livestock, vegetable, fish, fruits, life elixir, water and H3O2 structured water and cosmetic products.

### Advantageous Effects of Invention

In the last 50 years, rehabilitation of patients with CP and MS, various types of diseases like cancers, Parkinson's, diabetes and AIDS and various types of infertility has been developed. One of the most important problems is that the artificial human waveforms called technology carry destructive information that lead cellular and neurological insanity that the body's defense system is virtually unavailable and unable to deal with them. At low doses, the body cells can restore many of the injuries inflicted by the body by radiation. At doses greater than 100 mSv/h the protective mechanisms of cells do not have the chance to repair all the injuries. In this case, the severity of the effects increases nonlinearly and causes the patient to develop mental and physical illnesses.

One of the most powerful and effective solutions to this problem is “Frequency Feng Shui with Cosmic Waves” which carries basic information called the Vitality force, provided that these waves are trapped and exploited for the health of the human being.

Some of these advantages and goals are:

Rehabilitation of all types of CP, MS and Parkinson's diseases

Treating various types of cancer and illnesses associated with cellular intelligence, including AIDS

Treating various neuronal neurological diseases

Reducing the duration of treatment after all types of surgeries and transplantation

Trapping cosmic waves for more than an hour in a quantum pyramid cavity and benefiting from it for the treatment of physical and mental illnesses

Maximizing cosmic wave in the cavity up to 21 times without the presence of the patient

Maximizing cosmic wave in the cavity up to 50 times with the presence of the patient

Since these life based waves are the energy of all creatures, exposure to these waves has no harmful physiological and psychological effects

Molecular rehabilitation and youth renewal known as elixir of life

Modifying genetic codes in DNA

Resolving infertility crisis

Definitive treatment of diabetes

Treating waters with microbial contamination

Plants grow more rapidly and the nature of these plants is organic and energetic

Breeding livestock and all kinds of marine animals with an energetic approach to promote health and healthy nutrition

People become more relaxed

Increasing human thinking power

Usability for all ages

### BRIEF DESCRIPTION OF DRAWINGS

The drawing figures depict one or more implementations in accord with the present teachings, by way of example only, not by way of limitation. In the figures, like reference numerals refer to the same or similar elements.

FIG. 1

FIG. 1 is a exploded pyramid, consistent with one or more exemplary embodiments of the present disclosure.

FIG. 2

FIG. 2 is the assembled façade with a complete view of the pyramid, consistent with one or more exemplary embodiments of the present disclosure.

FIG. 3

FIG. 3 is the 2D section of the pyramid from the angle of entrance door, consistent with one or more exemplary embodiments of the present disclosure.

FIG. 4

FIG. 4 is the 2D section of the pyramid from other sides, consistent with one or more exemplary embodiments of the present disclosure.

FIG. 5

FIG. 5 is the 3D image of pyramid, chassis, fiber, the Al base sheet, water, crystal and crystal chair, consistent with one or more exemplary embodiments of the present disclosure.

FIG. 6

FIG. 6 is the 3D image of the lower half of the exploded pyramid (Al section), consistent with one or more exemplary embodiments of the present disclosure.

FIG. 7

FIG. 7 is the 3D image of the upper half of the exploded pyramid (Cu section), consistent with one or more exemplary embodiments of the present disclosure.

FIG. 8

FIG. 8 is a Pyramid Regulator Antenna, consistent with one or more exemplary embodiments of the present disclosure.

FIG. 9

FIG. 9 is a flowchart showing the steps involved in energizing materials including water or anything that is formed of water, consistent with one or more exemplary embodiments of the present disclosure.

FIG. 10

FIG. 10 is a Flowcharts of the direct method of human exposure to cosmic waves, consistent with one or more exemplary embodiments of the present disclosure.

FIG. 11

FIG. 11 is a COMSOL output on the amplification of the input wavelength from 21 to 50 times of the maximum field due to the similar neuronal resonance of the brain with cosmic input waves, consistent with one or more exemplary embodiments of the present disclosure.

FIG. 12

FIG. 12 is a COMSOL output on the amplification of the input wavelength up to 21 times and displaying three spherical maxima of the pyramid, consistent with one or more exemplary embodiments of the present disclosure.

FIG. 13

FIG. 13 is a another output of the COMSOL software based on displaying the cosmic waves inside the pyramid, consistent with one or more exemplary embodiments of the present disclosure.

FIG. 14

FIG. 14 is a another output of the COMSOL software based on the maxima graph in the amplified vertical field by 21 times relative to the initial input, consistent with one or more exemplary embodiments of the present disclosure.

FIG. 15

FIG. 15 is a another output of the COMSOL software based on displaying the cosmic waves inside the pyramid, consistent with one or more exemplary embodiments of the present disclosure.

FIG. 16

FIG. 16 is a matching and coordinating the cortex wavelength and brain neuron with cosmic wavelength and the similarity to prove the image of “connecting cosmic intelligence to neuronal intelligence and cellular intelligence”, consistent with one or more exemplary embodiments of the present disclosure.

FIG. 17

FIG. 17 is an existing values of wavelengths from radio to cosmic waves, consistent with one or more exemplary embodiments of the present disclosure.

FIG. 18

FIG. 18 is the etheric body layer scanned by the Bio-well kirlian device

“Connecting the etheric body to the Cosmic Intelligence Network”, consistent with one or more exemplary embodiments of the present disclosure.

FIG. 19

FIG. 19 is the path of meridians in terms of acupuncture medicine, consistent with one or more exemplary embodiments of the present disclosure.

FIG. 20

FIG. 20 is a monitoring the balance of homeostasis in Chakra in the meridian circuit of human body by Bio-well kirlian device, consistent with one or more exemplary embodiments of the present disclosure.

FIG. 21

FIG. 21 is a Cosmic Health Land, consistent with one or more exemplary embodiments of the present disclosure.

### DETAILED DESCRIPTION OF THE DRAWINGS

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

For purposes of reference, it should be understood that the techniques and systems disclosed herein are applicable to coupled motion in a wrist; however, the techniques and systems may be adapted to a number of other applications . . . .

A cosmic resonator with the ability to trap cosmic waves acts as a quantum trap that absorbs and enhances resonance and increases the life span of input cosmic waves.

The input cosmic waves enter the pyramid vertex through a pyramid regulator antenna 001 to centralize the wave at the maximum of 057, 058, and 059 and to regulate the cosmic waves. Then, a collector flange 002 is used that prevents vortices in the inlet and facilitates assembly of “Input wave unity”. The collector flange 003 is connected to four copper edges 004 by some pins. This corner edge 004 in the upper half is responsible to transmit cosmic waves from the pyramid vertex to the lower half. Then using some pins, the copper corner edge 005 is connected to four aluminum corner edges 006 and the upper copper corner edges 004 are connected to the lower aluminum corner edges 006. In the lower half, the silver coating on the aluminum 021 is used to enhance the reflection coefficient and the surface plasmon of the silver coating on the aluminum base. The upper made of arsenide gallium instead of copper.

The task of four aluminum corner edges 006 in the lower part is to transmit the wave to the aluminum base 015 at the bottom, increase, and maintain the reflection of cosmic waves at three maximum values of 57, 58 and 59. It is fixed with four 007 holders by the holder fixing screw 022 the aluminum edges 006 are fixed on the aluminum base 015 at the bottom. The aluminum base 015 at the bottom is above the insulating fiber 016 and it is insulated against the bottom chassis 017. The aluminum base 015 at the bottom is responsible to maintain the cosmic waves in the pyramid.

The upper half has four copper coatings 008 acting as the cover and absorber of the wave. There are four aluminum coatings 009 acting as the cover and maintainer of the wave in the lower half. The entry to the structure with door 010 is embedded on one of the coatings 008 and 009.

Aluminum parts 006,012 are used with a number of screws of aluminum 011 to fix the aluminum parts 006 and 012 and the aluminum plate 012, which is responsible for maintaining the water of the floor 018 at the bottom of the pyramid, is used to increase the resilience and resonance coefficient at the bottom of the pyramid. At the top of the water 018 the bottom crystal glass 013 is located that the pure crystal glass 013 is used to prevent the wave failure provide the ability to move into the pyramid and exposure to cosmic waves. There is pure crystal chair 019 above it for human presence that does not cause the deflection of cosmic waves. It is done with a number of base cylindrical glass cylinders 014 for installation of pure crystal glass 013 one these bases 014.

The chassis 017 is the main structure and the pyramid is fixed on this chassis 017, above which 017 insulation fiber 016 is placed on that main aluminum base 015 and insulates chassis 017 against the base aluminum 015.

The orb 020 is used to produce high life elixir, structural water H3O2 and cosmetic energy products. At its maximum, the pyramid vertex 57 is used as auxiliary equipment.

The scale of the present invention is 1, 10 and 100 scale, which has the absorption feature, increases resonance and durability of input cosmic waves with the aim of converting energy and dark matter, benefiting from frequency wavelengths and quantum space information. One of its outputs is loading the Vitality Force data on water or any water-based living creature or substance and direct production and utilization of energetic products to enhance human and water health.

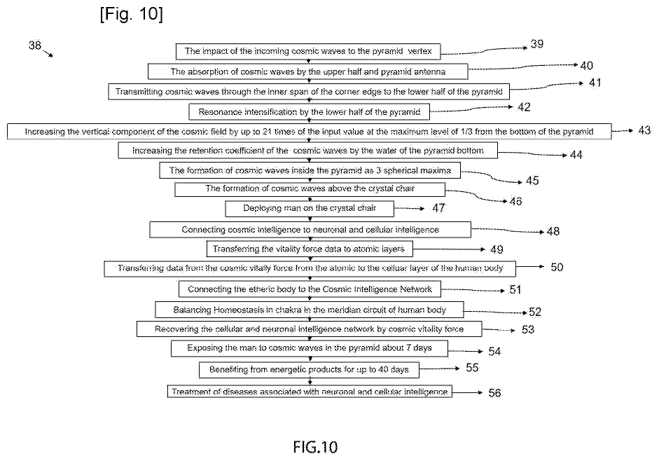

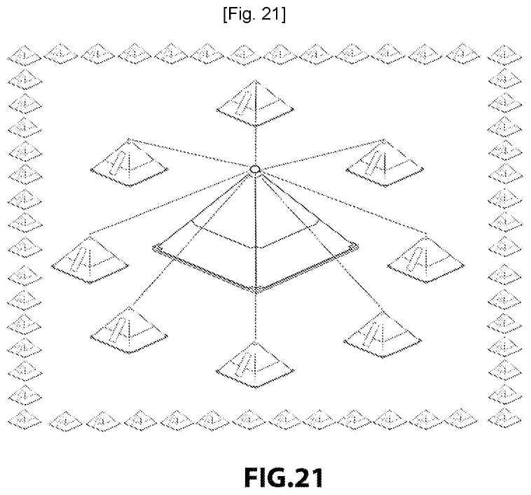

This structure is simulated with dimensions and capacities of 1, 10 and 100 scaleti of the cosmic resonator, which conforms to the dimensions of the structure and has the same result at 1 and 10 scales as cosmic health land FIG. 21 designed to make the products energetic FIG. 9 and directly expose man cosmic waves FIG. 10.

The simulated pyramid set is titled Cosmic Health Land FIG. 21 with 1 or 10 or 100 scale that have a holographic connection with each other. The bottom of the pyramid is to bath the patient resulting in complete health. The side pyramid with a scale of 1, 10 is to achieve the goals of FIG. 9 i.e. energetic water, energetic cosmetic products, life elixir, genetic modification, brain enhancement with cosmic waves, fish and livestock breeding in energetic cosmic environment, growing vegetables with hydroponic culture method and loading cosmic energy on a variety of fruits and dairy products.

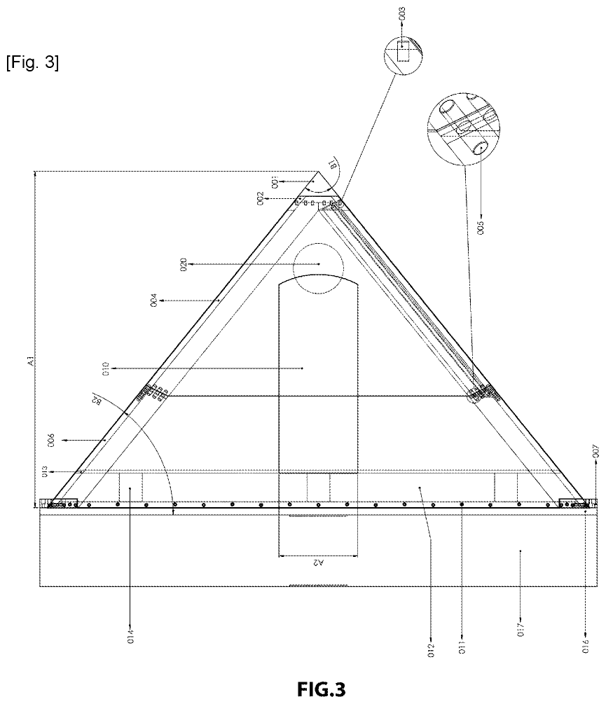

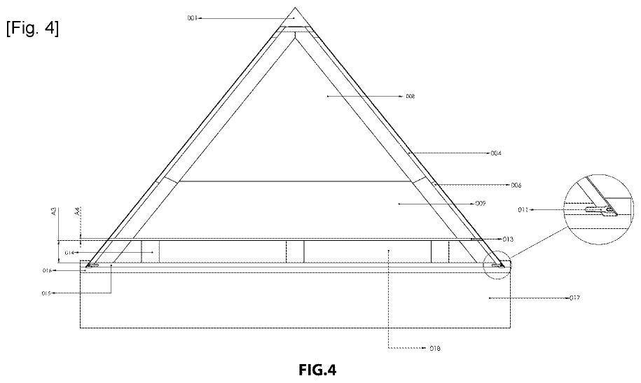

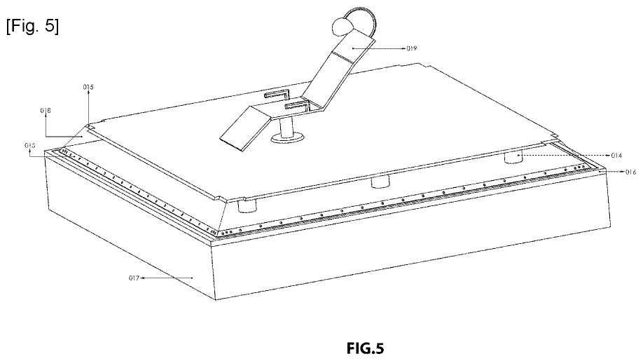

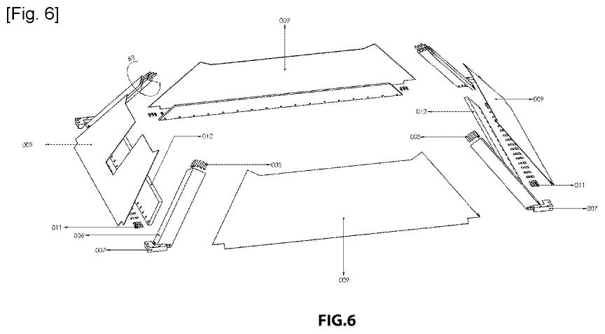

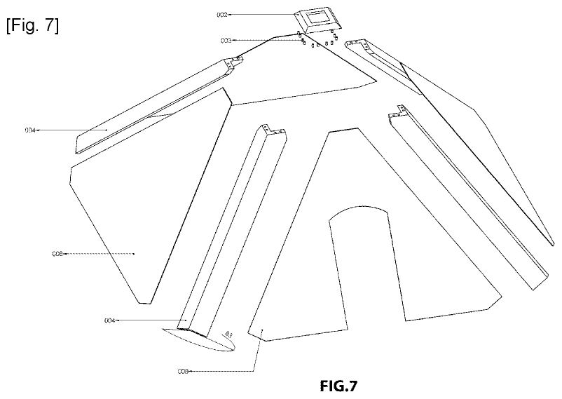

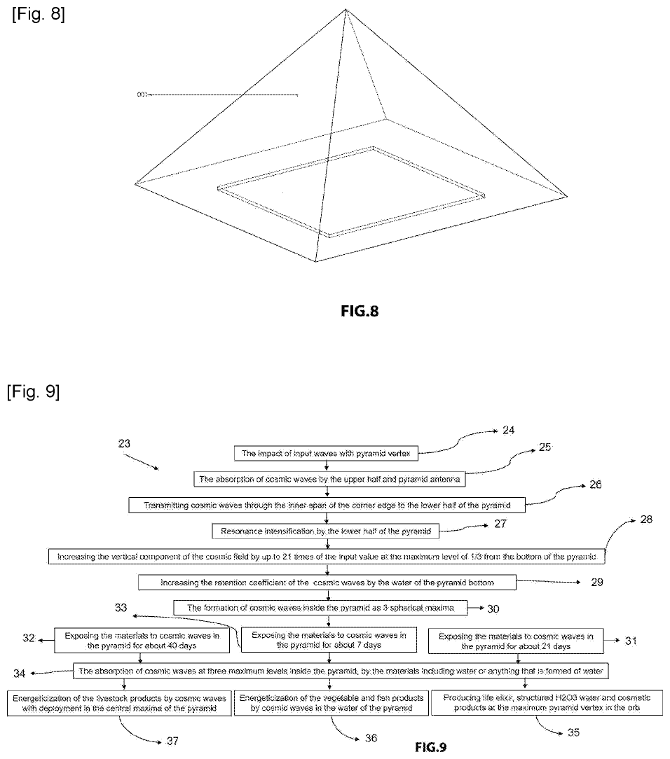

In FIG. 3, B1 is Pyramid vertex angle, B2 is The aluminum edge angle to the 2D aluminum base. in FIG. 6, B3 is The internal span angle between the copper corner edge and the aluminum corner edge.

The method of energeticization of materials, including water or whatever is formed of water 023 in FIG. 9 contains the following steps:

The impact of input waves with pyramid vertex 024

The absorption of cosmic waves by the upper half and pyramid antenna 025

Transmitting cosmic waves through the inner span of the corner edge 004 to the lower half of the pyramid 026

Resonance intensification by the lower half of the pyramid 027

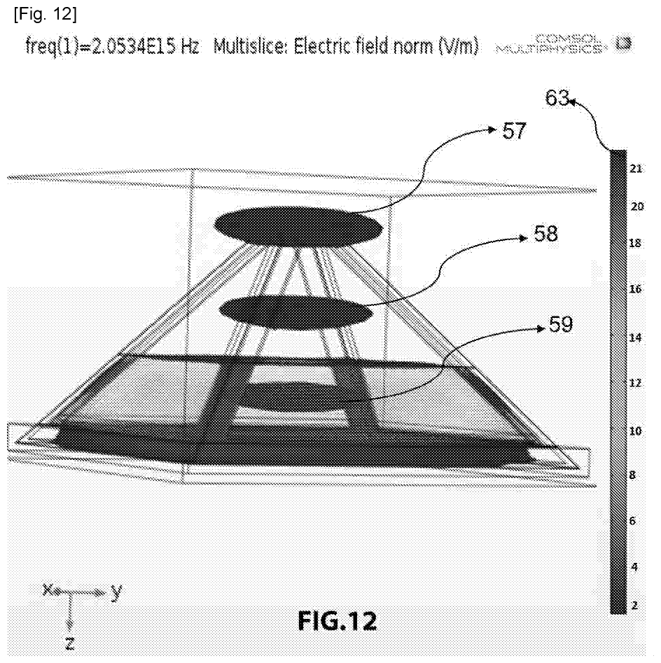

Increasing the vertical component of the cosmic field by up to 21 times of the input value FIG. 12 at the maximum level of 1/3 from the bottom of the pyramid 028

Increasing the retention coefficient of the cosmic waves by the water of the pyramid bottom 018-029

The formation of cosmic waves inside the pyramid as 3 spherical maxima 57, 58 and 59—FIG. 12—as 030

Absorbing cosmic waves at three maximum levels inside the pyramid, by the materials including water or anything that is formed of water 034

Exposing the materials to cosmic waves in the pyramid for about 40 days to energize livestock products by cosmic waves with deployment in the central maxima of the pyramid 035

Exposing the materials to cosmic waves in the pyramid for about 21 days to energize vegetable and fish products by cosmic waves in the water of the pyramid 036

Exposing the materials to cosmic waves in the pyramid for about 7 days to produce life elixir, structured H3O2 water and cosmetic products at the maximum pyramid vertex in the orb 037

According to FIG. 21, the pyramids, on the scales of 1 or 10 or 100 establishes a holographic relationship at these scales in cosmic health land, which is presented in parallel and simultaneously in the field of direct human exposure to waves and the use of energetic products.

In direct human exposure to cosmic waves, 038 in FIG. 10, in the first stage, the collision of the cosmic to the pyramid vertex 039 occurs and then cosmic waves are absorbed by the upper half and pyramid antenna regulator 040.

Transmitting cosmic waves through the inner span of the corner edge 004 to the lower half of the pyramid 041

Resonance intensification by the lower half of the pyramid 042

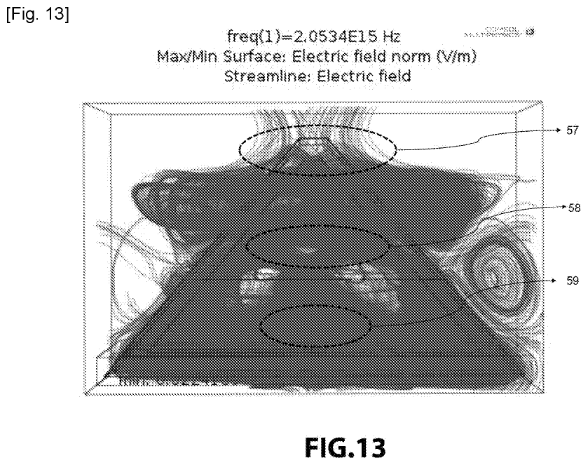

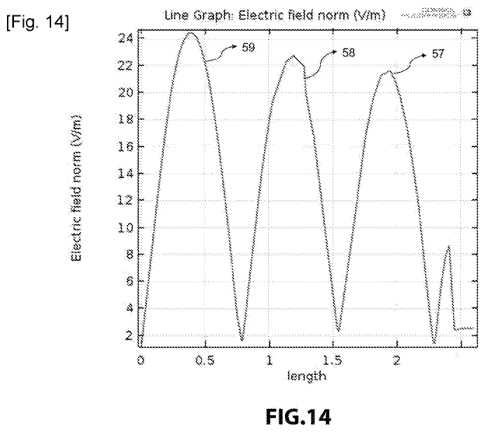

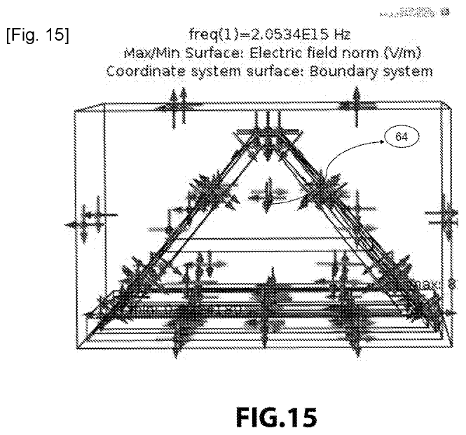

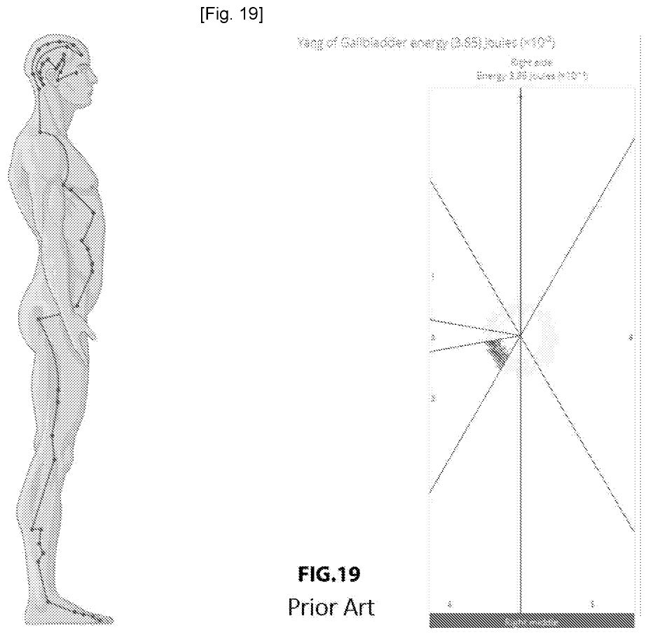

Increasing the vertical component of the cosmic field by up to 21 times of the input value at the maximum level of 1/3 from the bottom of the pyramid FIG. 12-043

Increasing the retention coefficient of the cosmic waves by the water of the pyramid bottom 044

The formation of cosmic waves inside the pyramid as 3 spherical maxima 57, 58 and 59—FIG. 12—045

The formation of cosmic waves above the crystal chair 046

Deploying man on the crystal chair 047 (as standing or sitting)

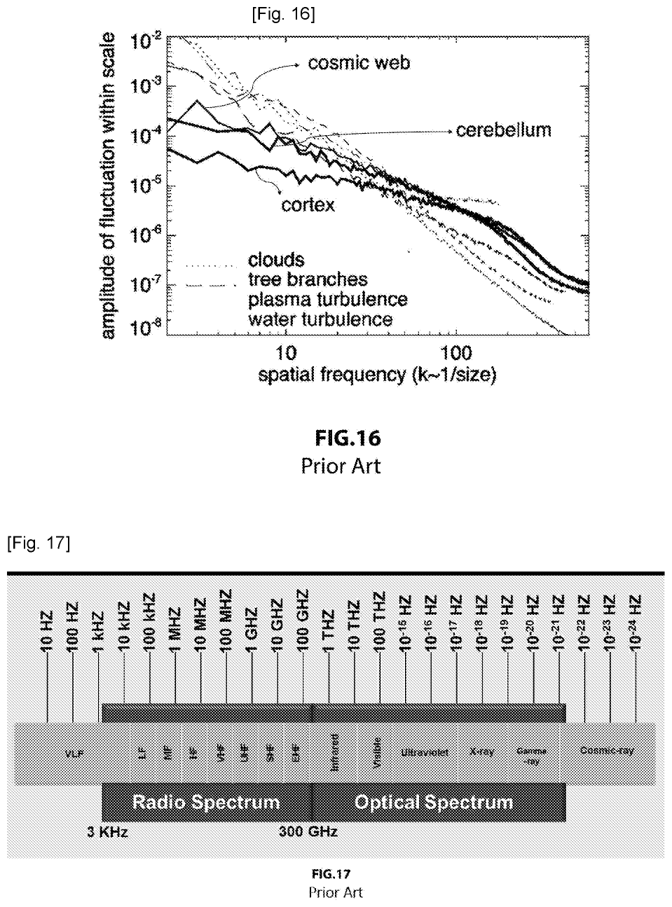

Connecting cosmic intelligence to neuronal and cellular intelligence 048—FIG. 16

Connecting cosmic intelligence to neuronal and cellular intelligence 049

Transferring data from the cosmic vitality force from the atomic to the cellular layer of the human body 050

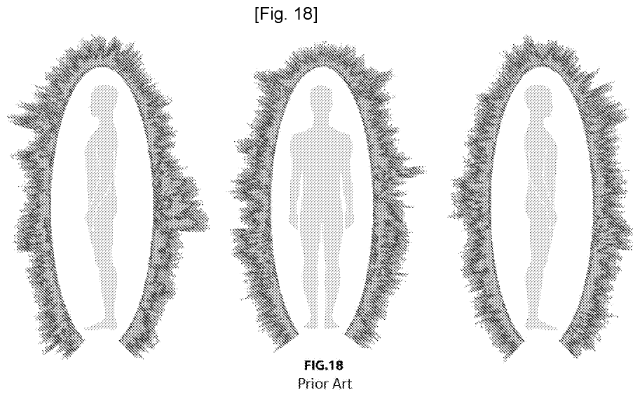

Connecting the etheric body to the Cosmic Intelligence Network FIGS. 18—051

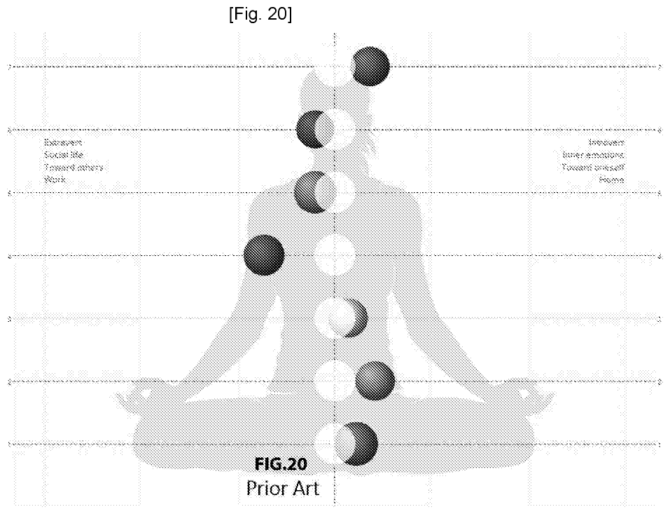

Balancing homeostasis in chakra in the meridian circuit of human body FIG. 20-052

Recovering the cellular and neuronal intelligence network by cosmic vitality force 053

Exposing the man to cosmic waves in the pyramid for about 7 days 054

Benefiting from energetic products for up to 40 days 055

Treatment of diseases associated with neuronal and cellular intelligence 056

In terms of dimensions, angles and edges, it is designed according to the effective cosmic wavelength that intensifies and collects the field inside the structure. The specification of this structure is based on the solution of field equations and is approved by the COMSOL software

The electric field of the flat wave in Z direction with the linear polarization of the cosmic ray is considered as follows:

E(x·y·z)=E(x·y)e^{-ik}^{_{z}}^{z} [math.1]

The wave equation can be written as follows:

$$∇ \times ( \frac{∇ \times E}{{μ}_{r}} ) - {k}_{0}^{2} {ɛ}_{r} E = 0 \qquad [ math . 2 ]$$

The wave vector in the free space is equal to:

$${k}_{0} = ω \sqrt{{ɛ}_{0} {μ}_{0}} = \frac{ω}{{c}_{0}} \qquad [ math . 3 ]$$

Where, is the speed of light in vacuum. The wave equation can be written as follows:

$$( ∇ - i {k}_{z} z ) \times [ \frac{( ∇ - i {k}_{z} z ) \times E}{{μ}_{r}} ] - {k}_{0}^{2} {ɛ}_{r} E = 0 \qquad [ math . 4 ]$$

Where z is the unit vector in the z direction.

The boundary conditions for the electric field are considered as follows:

n×E=n×E₀ [math.5]

The boundary between the imaginary chamber surfaces where the modeling is transient for the output wave.

E=E_{sc}e^{-ik(n·r)}+E₀e^{-ik(k·r)} [math.6]

Where E₀ is the electric field in the k direction for the input flat wave and E_{sc} is the propagated electric field.

The materials and alloys used in this structure have the least amount of waste at this wavelength. In order to focus the field in the structure, there is a need for sequential reflections inside the structure, which resembles an optical resonance cavity.

One of the simulated cases is in the 10⁹ to 10²⁰ ev range of these rays. The application of the reflection at 1.46×10⁻¹⁵ m wavelength is very difficult. Therefore, there is a need for a material with a coefficient of refraction (n) less than one, high electrical conductivity (σ), magnetic permeability (μr) close to one and low absorption coefficient (a) in this wavelength. Using the spectroscopy method, a hybrid alloy X1, X2 with a specific thickness was achieved and the resonance effect in this structure was provided. The simulation results show that the electric field's vertical component is 21 times greater than the initial value.

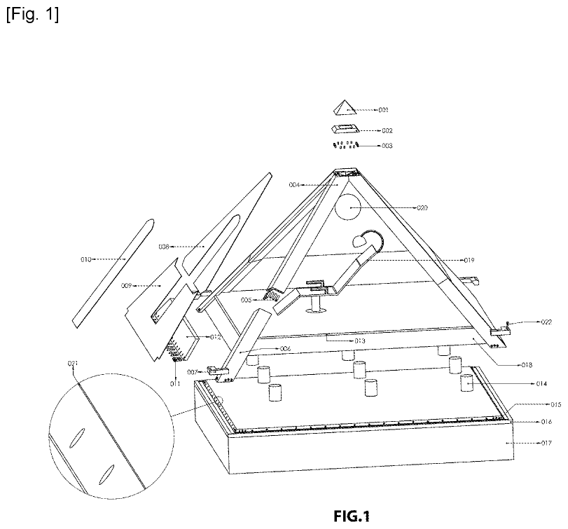

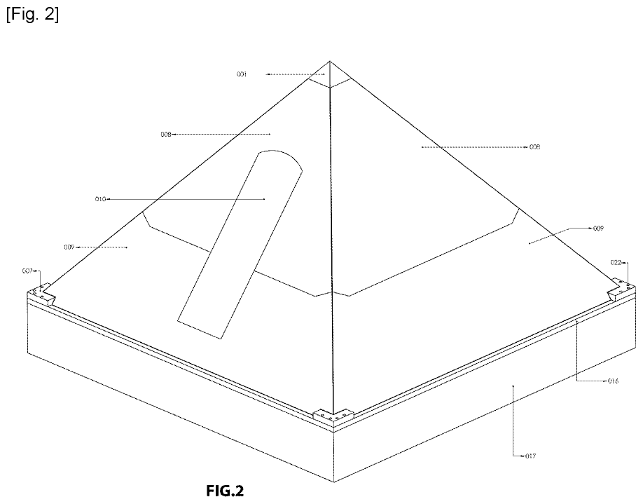

The dimensions and angles of FIG. 1 are based on the design and modeling of the COMSOL Multiphasic software. For the first time the smallest detected wavelength in the range of 10⁹ ev to 10²⁰ ev FIG. 017 is achieved by 21 times greater than the maximum 061—FIG. 12 with optical and reflective design and surface Plasmon analysis in various materials.

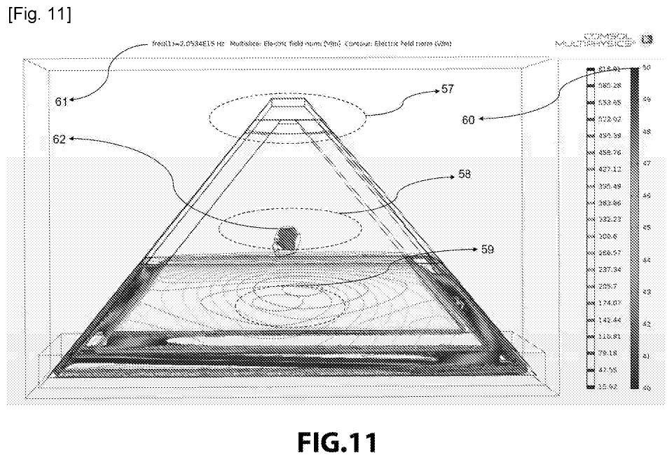

In a 21-fold increase of the vertical field FIG. 12—063 the presence of a nail or even a plastic chair will deviate the entire wavelength and in the same situation, with the simulation of the human head in the pyramid FIGS. 11, 13, 14 and 15—062 the efficacy of cosmic rays is simulated by controlling the field intensity and analyzing their absorption in the biology context.

In this invention, by investigating the direction of waves about the behavior of cosmic rays in the collision with different parts of the pyramid 064, their interactions with different materials and the effects of their field on physiological action the efficiency of these rays is achieved. According to the same resonance of the human head and cosmic waves FIG. 16, the 21 folded vertical component and wavelength 063 increased to 50 fold 060 and FIG. 16 also proved the similarity of cosmic wavelengths and brain cortex wavelength resonance.

Since pyramid quantum traps have been designed in this invention with optical details under the 763 simulation stage with COMSOL Multiphysics software and its industrial prototype has been built, the absorption durability and increase in resonance and the durability of cosmic input wavelengths are beneficial for human knowledge of space time information and vitality force to expand the boundaries of medical science, here example of energetic products in terms of human health are presented.

The process of producing energetic products by the said materials, including water or anything that is made of water and human exposure to cosmic waves is through simultaneous and parallel deployment of 1, 10 and 100 folded pyramids alongside in the construction of a place called cosmic health land as FIG. 21.

Energizing the materials FIG. 9 based on the absorption of cosmic waves at three maxima inside the pyramid by the mentioned materials, including water or any water-containing material, including the construction of a cosmic health land FIG. 21 and the establishment of a pyramid at 1, 10 and 100 scale to achieve the following products;

Energizing the livestock products by cosmic waves by deploying them at the center of the pyramid to expose materials to cosmic waves in the pyramid for about 40 days

Producing energetic water in order to maintain human health and benefit from meat and dairy products to achieve physical and mental health

Producing energetic fish by the cosmic waves in the water of the pyramid for 21 days in the form of fish culture in energetic water

Production of energy plants by cosmic waves in the water of the pyramid for 21 days in the form of culturing plants inside the water of the pyramid

Production of vegetables in the water of the pyramid and the human use of these energetic and cosmetic products for physical and mental health

The production of life elixir and structural H3O2 at the pyramid vertex within the spherical orb for 7 days for cellular rejuvenation

Producing energetic cosmetic products by exposing these products to cosmic waves for 7 days at the pyramid vertex inside the spherical orb and benefiting from it to eliminate skin damages

Production of organic and energetic fertilizer

The presence of humans in the maximum of one-third of the pyramid is due to physical and psychological health FIG. 10 and frequency feng shui due to cosmic waves.

Human bath in the energetic water at the bottom of pyramid with 1, 10, 100 scale FIG. 21 and connecting the body to vitality force and treatment of illnesses related to cellular and neuronal intelligence as well as loading vital information on the subatomic structure of materials including water and anything formed of water for human health in the form of homeostasis energy balance. Connecting the etheric body to the cosmic intelligence network, which results in treating mental and neuronal intelligence illnesses and all illnesses related to cellular intelligence and reducing the length of treatment after a variety of transplantational surgeries in humans.

Improvement of the efficiency of the brain through the co-resonance of neuronal wavelength with cosmic waves resulting in the release of DMT from the Pineal gland.

Modified Gene Production

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 202, 204, or 206 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study, except where specific meanings have otherwise been set forth herein. Relational terms such as “first” and “second” and the like may be used solely to distinguish one entity or action from another without necessarily requiring or implying any actual such relationship or order between such entities or actions. The terms “comprises,” “comprising,” or any other variation thereof, as used herein and in the appended claims are intended to cover a non-exclusive inclusion, encompassing a process, method, article, or apparatus that comprises a list of elements that does not include only those elements but may include other elements not expressly listed to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is not intended to be used to interpret or limit the scope or meaning of the claims. In addition, in the foregoing Detailed Description, it can be seen that various features are grouped together in various implementations. Such grouping is for purposes of streamlining this disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into this Detailed Description, with each claim standing on its own as a separately claimed subject matter.

While various implementations have been described, the description is intended to be exemplary, rather than limiting and it will be apparent to those of ordinary skill in the art that many more implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

### EXAMPLES

According to one or more exemplary embodiments of the present disclosure, Since pyramid quantum traps have been designed in this invention with optical details under the 763 simulation stage with COMSOL Multiphysics software and its industrial prototype has been built, the absorption durability and increase in resonance and the durability of cosmic input wavelengths are beneficial for human knowledge of space-time information and vitality force to expand the boundaries of medical science. The Cosmic Health Land has been designed and registered to create health tourism pyramid complexes.

Cosmic Health Land is based on energizing materials based on the absorption of cosmic waves at three maxima inside the pyramid by the mentioned materials, including water or any other water-based product. The purposes of establishing Cosmic Health Land at 1: 1, 10 and 100 scales is to achieve the following goals

Energizing the livestock products by cosmic waves by deploying them at the center of the pyramid to expose materials to cosmic waves in the pyramid for about 40 days

Energizing fish and vegetables by the cosmic waves in the water of the pyramid for 21 days in the form of fish culture in energetic water

Growing vegetables with hydroponic culture method and in the energetic water of the pyramid bottom and using the cosmic and energetic products for mental and physical health

Producing life elixir and structural H3O2 at the pyramid vertex within the spherical orb for 7 days for cellular rejuvenation

Energizing energetic cosmetic products by exposing these products to cosmic waves for 7 days at the pyramid vertex inside the spherical orb and benefiting from it to eliminate skin damages

Breeding livestock in the pyramid and energizing animal products with cosmic waves by exposing at the center of the pyramid for 40 days and benefiting from the meat and dairy in order to achieve physical and mental health

Producing energetic water to maintain human health

The presence of humans in the maximum of one-third of the pyramid is due to physical and psychological health FIG. 10 and frequency feng shui due to cosmic waves

Human bath in the energetic water at the bottom of pyramid with 1, 10 and 100 scale and connecting the body to vitality force and treatment of illnesses related to cellular and neuronal intelligence as well as loading vital information on the subatomic structure of materials including water and anything formed of water for human health in the form of homeostasis energy balance. Connecting the etheric body to the cosmic intelligence network, which results in treating mental and neuronal intelligence illnesses and all illnesses related to cellular intelligence and reducing the length of treatment after a variety of transplantational surgeries in humans.

### INDUSTRIAL APPLICABILITY

The presentation method is as energy products, and the production and sale of pyramid at specified scales and the provision of services as a project in the form of the implementation of a set of pyramid networks as Cosmic Health Land at 1, 10 and 100 scales.

Energizing the animal products by cosmic waves

Producing energetic water to maintain human health

Producing energetic fish

Producing energetic vegetables

Producing energetic vegetables in the energetic water of the pyramid bottom

Producing life elixir and structure H3O2

Producing energetic cosmetic products

Producing organic and energetic fertilizer

The presence of humans in the maximum of one-third of the pyramid is due to physical and psychological health (FIG. 10) and frequency feng shui due to cosmic waves

Human bath in the energetic water at the bottom of pyramid with 1, 10 and 100 scales

Improvement of the efficiency of the brain through the co-resonance of neuronal wavelength with cosmic waves resulting in the release of DMT from the Pineal gland.

### CITATION LIST

Citation List follows:

### Patent Literature

PTL1: EP Application, EP19800400320
PTL2: CN patent, CN1617188
PTL3: DE application, DE102007010424
PTL4: PCT application, WO2015022697A1

1. A cosmic resonator is Pyramid cosmic resonator with the capability to capture cosmic waves acting as a quantum trap that absorbs and enhances resonance and increases the life span of input cosmic waves. It is formed with the aim of loading vitality force information on water or any water-based material and uses it to promote human and water health directly or indirectly, comprising:
a aluminum base at the bottom that is responsible to maintain cosmic waves in the pyramid;
water of the pyramid bottom by Increasing the retention coefficient of the pyramid;
a lower half whose purpose is to enhance resonance, promote retention coefficient, and increase durability of the incoming cosmic waves, wherein the structure comprises:
Four aluminum corner edges connected to the ends of the base aluminum and along the edges of the aluminum along the pyramid vertex on both sides; and
An aluminum plate to keep water at the bottom of the pyramid; and
a upper half that absorbs cosmic waves, wherein the structure comprises:
Four copper corner edges to transmit wave from the vertex to the bottom of the pyramid, retain, and reflect waves at the maxima formed at the collision of the aluminum edges along the pyramid vertex.

2. The cosmic resonator as claimed in claim 1, wherein the lower half, the crystal chair is placed to deploy the man on the crystal glass floor.

3. The cosmic resonator as claimed in claim 1, wherein the base aluminum at the bottom is attached to the crystal by the crystal glass base to prevent failure and distortion of the waves above the aluminum.

4. The cosmic resonator as claimed in claim 1, wherein there is insulating fiber beneath the aluminum and an iron square chassis under the insulating fiber to fix the structure.

5. The cosmic resonator as claimed in claim 1, wherein, the aluminum corner edge is coated with aluminum.

6. The cosmic resonator as claimed in claim 1, wherein the copper corner edge is coated with copper.

7. The cosmic resonator as claimed in claim 1, wherein a hollow orb is placed at one third of the top of the pyramid to supply life elixir, structured H3o2 and energetic cosmetic products.

8. The cosmic resonator as claimed in claim 1, wherein the aluminum base is coated with silver.

9. The cosmic resonator as claimed in claim 1, wherein the lower half, the aluminum base is connected to the bottom crystal with at least one crystal base at the lower half.

10. The cosmic resonator as claimed in claim 1, wherein the lower half, there are aluminum screws to fix the Al plate and Al corner edge to the Al base.

11. The cosmic resonator as claimed in claim 1, wherein the upper half, there are Cu screws to connect Cu corner edge to Al corner edge.

12. The cosmic resonator as claimed in claim 1, wherein the upper half, upper the upper half is a pyramid regular antenna to concentrate and fix cosmic waves at three internal maxima and set the intensity of the input waves.

13. The cosmic resonator as claimed in claim 12, wherein there is a collector flange beneath the antenna to prevent vortices in the cavity input that connects the upper half to the antenna.

14. The cosmic resonator as claimed in claim 12, wherein some screws connect the collector flange to the 4 Cu corner edges.

15. The cosmic resonator as claimed in claim 1, wherein the lower half, the entrance could be located among each side of the pyramid.

16. The cosmic resonator as claimed in claims 1, 7, wherein the pieces made of alumina can be made of silver.

17. The cosmic resonator as claimed in claim 1, wherein the pieces made of Cu can be made of arsenide gallium.

18. The cosmic resonator as claimed in claim 1, wherein in order to prevent the shear stress, the bottom of the Aluminum corner edge is fixed to the Al base with holder.

19. The cosmic resonator as claimed in claim 1, wherein the energy of the waves received by the upper half or the antenna is in the range of about 10⁸ ev to about 10²⁰ ev, the frequency of the received wavelength is in the range of about 10²¹HZ to about 10ⁿ HZ and the wavelength of the received waves is in the range of about 10-22 [symbol] m to about 10-12 [symbol] m.

20. The cosmic resonator as claimed in claim 1, wherein the thickness of the insulation fiber is at least about 50 mm in diameter to insulate the Al base against chassis.

21. The cosmic resonator as claimed in claim 1, wherein the area of the chassis is about 16 square meters.

22. The cosmic resonator as claimed in claim 1, wherein the Al corner edge are made of AL 1000 to AL 7000, with a length of about 1200 mm to about 1400 mm, the width of about 180 mm to 250 mm, the thickness of about 35 mm to about 45 mm, the internal span angle of about 110° to about 115°.

23. The cosmic resonator as claimed in claim 1, wherein the Al base is square-shaped made of AL 1000 to AL 7000, with a length of about 3550 mm to 3850 mm, the width of 3550 mm to about 3850 mm, the thickness of about 35 mm to about 45 mm, the internal span angle of about 49° to about 53°.

24. The cosmic resonator as claimed in claim 1, wherein the Al base is connected and insulated to the chassis by pure aluminum beads made of AL 1000 to AL 7000 to maintain flatness of Al.

25. The cosmic resonator as claimed in claim 1, wherein the bottom water in the pyramid above the Al base has a height of about 150 mm to about 200 mm.

26. The cosmic resonator as claimed in claim 1, wherein the four Cu corner edges at the upper half of pyramid have the purity of 95 to 99.99, with a length of 1900 mm to 2100 mm, the width of 180 mm to 250 mm, the thickness of 35 mm to 45 mm, the internal span angle of about 110° to about 115°.

27. The cosmic resonator as claimed in claim 1, wherein the collector flange has Cu material with the purity of 95 to 99.99, gold coating with the thickness of about 0.5 mm to about 0.1 mm due to the union of the inlet wave and fixing the top of the Cu edges that with a length of about 340 mm to about 345 mm, the width of about 340 to about 345 mm and the thickness of about 42 mm to about 47 mm.

28. The cosmic resonator as claimed in claim 1, wherein the regulator antenna is solid made of Cu material with the purity of 95 to 99.99 and thickness of about 0.5 to about 0.1 mm; the thickness of solver coating is about 0.5 to about 0.1 mm; the thickness of gold coating based on the pyramid dimensions (length of about 270 mm to about 300 mm, the width of about 270 mm to about 300 mm and the height of about 150 mm to about 200 mm) and the internal span of the pyramid vertex is in the range of about 75° to about 80°.

29. The cosmic resonator as claimed in claim 1, wherein the crystal glass at the bottom is of pure crystal to prevent failure and wave deviation height of 170 mm to 220 mm vs. the aluminum base to allow deployment and movement of human in the pyramid. The dimensions of crystal glass are the length of about 3200 mm to about 3400 mm, the width of 3200 mm to about 3400 mm and the thickness of about 10 mm to about 20 mm.

30. The cosmic resonator as claimed in claim 1, wherein aluminum coated sheets in the lower half section are AL1000 to AL7000 and the coated sheets of copper in the upper half have a purity of about 95 to about 99.99 with a thickness of about 3 mm to about 5 mm.

31. The cosmic resonator as claimed in claim 1, wherein the aluminum plate is AL1000 to AL7000 with length of 3200 mm to 3400 mm, the width of 230 mm to 250 mm and the thickness of 35 mm to 45 mm and the internal span angle of 49° to 53°.

32. The cosmic resonator as claimed in claim 1, wherein the hollow orb is silver with a diameter of 500 mm to 600 mm and a thickness of 10 mm to 15 mm, and the gold coating has a thickness of 0.5 mm to 1 mm.

33. The cosmic resonator as claimed in claim 1, wherein the pyramid antenna is solid and removable that is placed on the flange.

34. The cosmic resonator as claimed in claim 1, wherein the direction of the entrance door is towards the north.

35. The cosmic resonator as claimed in claim 1, wherein by putting least one pyramid together in parallel with simultaneous production of energetic and the presence of humans exposed to cosmic waves simultaneously by simultaneous deployment of the 1: 1, 10, and 100 scale pyramids, a place called Cosmic Health Land is created.

36. The cosmic resonator as claimed in claim 1, wherein the useful height of the pyramid of the cosmic resonator from the pyramid vertex to the insulating fiber is about 2000 mm to about 2500 mm.

37. The cosmic resonator as claimed in claims 1 to 36, wherein all dimensions and scales of the sides and angles of the pyramid can be 1, 10, and 100 scale greater than the current sizes.

38. The method of energizing materials comprise of water or any other water-based material, said method comprising steps of:
impacting of input waves with pyramid vertex;
absorbing of cosmic waves by the upper half and pyramid antenna;
Transmitting cosmic waves through the inner span of the corner edge to the lower half of the pyramid;
Resonance intensification by the lower half of the pyramid;
Increasing the vertical component of the cosmic field by up to 21 times of the input value at the maximum level of 1/3 from the bottom of the pyramid;
Increasing the retention coefficient of the cosmic waves by the water of the pyramid bottom;
Exposing the materials to cosmic waves in the pyramid until about 40 days;
absorption of cosmic waves at three maximum levels inside the pyramid, by the materials including water or anything that is formed of water; and
Energizing the materials by cosmic waves.

39. The method as claimed in claim 38, wherein after the phase of increasing the cosmic wave's retention coefficient by the water, the formation of cosmic waves in the pyramid can occur as three spherical maxima.

40. The method as claimed in claim 38, wherein the exposure period of until 40 days is 7 days for life elixir, structured H3o2 and cosmetic products at the maxima of pyramid vertex inside the orb, 21 days for the fish and vegetables in the water and 40 days for livestock products at the center of the pyramid.

41. The method as claimed in claim 38, wherein the mentioned materials could be H203 water, energetic water, energetic cosmetics, life elixir, energetic fish, energetic livestock, energetic dairy products, energetic vegetables, energetic fruits, modified gene and enhanced brain.

42. The method of direct human exposure to cosmic waves, said method comprising step of:
The impact of input waves with pyramid vertex;
The absorption of cosmic waves by the upper half and pyramid antenna;
Transmitting cosmic waves through the inner span of the corner edge to the lower half of the pyramid;
Resonance intensification by the lower half of the pyramid;
Increasing the vertical component of the cosmic field by up to 21 times of the input value at the maximum level of 1/3 from the bottom of the pyramid;
Increasing the retention coefficient of the cosmic waves by the water of the pyramid bottom;
The formation of cosmic waves inside the pyramid as 3 spherical maxima;
The formation of cosmic waves above the crystal chair;
Deploying man on the crystal chair;
Connecting cosmic intelligence to neuronal and cellular intelligence;
Transferring cosmic vitality forces to the human atomic layer;
Transferring data from the cosmic vitality force from the atomic to the cellular layer of the human body;
Connecting the etheric body to the Cosmic Intelligence Network;
Balancing homeostasis in chakra in the meridian circuit of human body;
Recovering the cellular and neuronal intelligence network by cosmic vitality force;
Exposing the man to cosmic waves in the pyramid for about 7 days; and
Treatment of diseases associated with neuronal and cellular intelligence.

43. The method as claimed in claim 42, wherein human beings are placed on the crystalline chair, humans can enter the pyramid before or after receiving the cosmic waves by the pyramid.

44. The method as claimed in claim 42, wherein neurologic and cellular intelligence-related illnesses can include MS, CP, Parkinson's disease, mental illness, and various illnesses associated with cellular intelligence, including types of cancer, diabetes, AIDS and the treatment period may be reduced after all types of transplant surgery in humans and genetic modification.

45. The method as claimed in claim 38, wherein in addition to human exposure to cosmic waves directly for up to about 7 days, human rehabilitation continues up to 40 days after the consumption of energetic products indirectly in the form of cellular regeneration.

46. The cosmic resonator and method as claimed in claims 1, 38, 42, wherein the establishment of the pyramid can be in the appropriate location with the input waves in the orbit of 0 to 63 degrees of the Earth, Arctic or Antarctic.
